(19) United States
(12) Patent Application Publication
(10) Pub. No.: US 2011/0020429 A1
(43) Pub. Date: Jan. 27, 2011

(54) USE OF PARTICLES COMPRISING AN ALCOHOL

(75) Inventors: Cecilia Leal Lauten, Oslo (NO); Karen Sibylia Rognvaldsson, Oslo (NO); Sigrid L. Fossheim, Oslo (NO); Esben A. Nilssen, Oslo (NO)

Correspondence Address: BIRCH STEWART KOLASCH & BIRCH, PO BOX 747, FALLS CHURCH, VA, 22040-0747, US

(73) Assignee: Epitarget AS, Oslo (NO)

(21) Appl. No.: 12/747,085

(22) PCT Filed: Dec. 9, 2008
(86) PCT No.: PCT/NO08/00438

§ 371 Date: Sep. 20, 2010

(30) Foreign Application Priority Data
Dec. 10, 2007 (NO) 20076379

(51) Int. Cl. A61K 9/127; A61K 9/14; A61P 35/00; C07C 31/02; A61K 31/045

(52) U.S. Cl. 424/450; 424/400; 568/840; 514/724

(57) ABSTRACT

Novel use of ultrasound sensitive drug carrying particles comprising an alcohol is disclosed, as well as products and methods thereof. The drug carrying particles accumulate in the diseased target tissue and efficiently release their payload upon ultrasound exposure.

### FIELD OF THE INVENTION

The present invention is related to use of particles comprising alcohol for controlled drug delivery and release within a defined volume in an animal. The invention also relates to acoustically sensitive drug carrying particles, e.g. liposomes, as well as compositions and methods thereof.

### BACKGROUND OF THE INVENTION

Lack of targeted drug delivery reduces the therapeutic-to-toxicity ratio thus limiting medical therapy. This limitation is particularly evident within oncology where systemic administration of cytostatic drugs affects all dividing cells imposing dose limitations. Hence, it exists a clear need for more efficient delivery of therapeutic drugs at the disease target with negligible toxicity to healthy tissue. This challenge has to a certain extent been accommodated by encapsulating drugs in a shell protecting healthy tissue en route to the diseased volume. Such protective shells may include a number of different colloidal particles such as liposomes or other lipid dispersions, and polymer particles. However, development of such drug delivery particles has faced two opposing challenges: efficient release of the encapsulated drug at the diseased site while maintaining slow non-specific degradation or passive diffusion in healthy tissue. At present, this constitutes the main challenge in drug delivery (Drummond, Meyer et al. 1999).

Ultrasound (US) has been suggested as a method to trigger specific drug release (Pitt, Husseini et al. 2004). This may allow the engineering of robust particles protecting healthy tissue while in circulation, accumulating in the diseased volume and releasing the payload on exposure to acoustic energy. Also, US is known to increase cell permeability thus providing a twofold effect: drug carrier disruption and increased intracellular drug uptake (Larina, Evers et al. 2005; Larina, Evers et al. 2005).

Currently, four main types of US responsive particles are known: micelles, gas-filled liposomes, microbubbles and liposomes. Micelles are non-covalently self-assembled particles typically formed by molecules containing one part which is water soluble and one that is fat soluble. The monomer aqueous solubility is typically in the mM range and at a critical concentration; micelles are formed shielding the fat soluble part from the aqueous phase. Micelle formation and disruption is therefore an equilibrium process controlled by concentration, making these particles rather unstable and less suitable for drug delivery. In addition, limited drug types can be encapsulated. Gas-filled liposomes and microbubbles are highly US responsive but too large (˜1 μm) for efficient accumulation in e.g. tumour tissue. In contrast, liposomes or other lipid dispersions may encapsulate a broad range of water soluble and fat soluble drugs, as well as efficiently accumulate in e.g. tumour tissue. However, reports on ultrasound sensitive liposomes are scarce.

Lin and Thomas (Lin and Thomas 2003) report that when liposome membranes are altered by the addition of phospholipid grafted polyethylene glycol (PEG-lipid) or non-ionic surfactants, the liposome is more responsive to US. The present applicant recently identified a synergistic interplay between liposomal PEG content and size with respect to US sensitivity (NO20071688 and NO20072822, incorporated herein by reference). Here, liposomes with both high PEG content and small size showed synergistically increased US responsivity or sonosensitivity and improved drug release properties.

Long-chain alcohols may also be incorporated in phospholipid bilayers. The alcohol has one part with affinity for water (hydroxyl group) and another with affinity for oily or lipidic environments (hydrocarbon moiety). When added to a liposome dispersion some alcohol molecules remain in the aqueous phase, whilst others are incorporated in the phospholipid membrane. The extent of incorporation depends on the alcohol chain length. The longer the chain length, the more molecules will be captured within the membrane (Aagaard, Kristensen et al. 2006). The fact that organic alcohols can penetrate membranes also has an implication on local and general anaesthesia in animals (Lee 1976).

The effect of alcohols on the liposomal membrane properties is remarkably different depending on the alcohol chain length. The membrane can be made “thinner” by inclusion of short chain alcohols (Rowe and Campion 1994; Tierney, Block et al. 2005) and the gel-to-liquid crystalline phase transition temperature of the membrane lowered by the addition of decanol (Thewalt and Cushley 1987). Interestingly, octanol which has a shorter chain is even more efficient to lower the phase transition temperature.

In a study conducted by the current applicant, it was shown for the first time that the antitumoural effect of liposomal doxorubicin (Caelyx®) could be enhanced when combined with ultrasound (Myhr and Moan 2006). However, liposomal doxorubicin (Caelyx® or Doxil®) is not engineered for ultrasound mediated drug release and shows a rather low release in vitro (see e.g. WO2008120998A2, incorporated herein by reference).

The current inventors disclose that the sonosensitivity of drug delivery particles is surprisingly improved by incorporation of alcohols into the membrane. From prior art, it is known that incorporation of ethanol improves skin penetration of liposomes (Barry 2001). In addition, alcohols are known to reduce liposomal uptake of the reticuloendothelial system (polyvinyl alcohol) or be of interest due to their emulgating or solubilising properties (lanolin alcohol and octadecanol). See e.g. WO 94/28873, WO A1 9428874, or U.S. Pat. No. 5,770,222, incorporated herein by reference. However, improved sonosensitivity by alcohol incorporation is neither shown nor suggested in prior art.

The current invention may be employed to efficiently deliver drugs in a defined tissue volume to combat localized diseases. Such particles may passively or actively accumulate in the target tissue and the drug payload may be dumped in the tissue by means of ultrasound thereby increasing the therapeutic-to-toxicity ratio.

### DEFINITIONS

DSPC herein means 1,2-distearoyl-sn-glycero-3 phosphocholine or, in short, distearoylphosphatidylcholine.

DSPE herein means 1,2-distearoyl-sn-glycero-3-phosphoethanolamine.

DSPE-PEGXXXX herein means 1,2-distearoyl-sn-glycero-3-phosphoethanolamine-N-[meth-oxy(polyethylene glycol)-XXXX, wherein XXXX signifies the molecular weight of the polyethylene glycol moiety, e.g. DSPE-PEG2000 or DSPE-PEG5000. If XXXX is omitted, the PEG may have any molecular weight.

n-alcohol means any alcohol with n carbon atoms.

PEG herein means polyethylene glycol or a derivate thereof.

PEGXXXX herein means polyethylene glycol or a derivate thereof, wherein XXXX signifies the molecular weight of the polyethylene glycol moiety.

PC herein means phosphatidylcholine with any composition of acyl chain.

PE herein means phosphatidylethanolamine with any composition of acyl chain.

‘US’ herein means ultrasound.

‘US sensitive’, ‘sonosensitive’ or ‘acoustically sensitive’ herein means the ability of an entity, e.g. a particle, to respond to acoustic energy of any frequency.

Nominal concentration herein means the initial (weighed amounts per given volume) concentration of a constituent in the liposome membrane or in the hydration medium.

### General Provisions

The phospholipid, cholesterol, PEG-lipid and hexanol liposome concentrations herein are nominal values unless otherwise stated.

In the current disclosure singular form means singular or plural. Hence, ‘a particle’ may mean one or several particles. Furthermore, all ranges mentioned herein includes the endpoints, that is, the range ‘from 14 to 18’ includes 14 and 18.

### DETAILED DESCRIPTION OF THE INVENTION

The current invention comprises a sonosensitive particulate material comprising an alcohol.

The particulate material may be arranged in any form of dispersion of a given internal structure. Examples of preferred structures are inverted hexagonal phases, like Hexosome® or Cubosomes®, emulsion, microemulsions, liquid crystalline particles and liposomes. According to a preferred embodiment, the particulate material is a membrane structure, more preferably a liposome.

The alcohol may be any alcohol, however, primary or secondary alcohols are preferred. The alcohol or primary alcohol may be any n-alcohol where n=2-20 carbon atoms; preferably n=2-10; more preferably propanol, butanol, hexanol, heptanol, or octanol, or any combination thereof; even more preferably hexanol, heptanol, or octanol, or any combination thereof. In a preferred embodiment of the current invention, the alcohol or primary alcohol is a hexanol. Any concentration of alcohol, e.g. hexanol, may be used in the hydration liquid used to hydrate the lipid film and generate liposomes. In general, more hexanol results in higher sonosensitivity. Accordingly, the nominal alcohol concentration is at least 1 mM, preferably at least 10 mM, more preferably above 25 mM, more preferably above 50 mM, even more preferably above 60 mM, and most preferably around 75 mM. The inventors prefer that the concentration is within the range 50 mM to 80 mM, more preferably within the range 60 mM to 75 mM. In embodiments of the current application the hexanol concentrations are 25, 50, 60, or 75 mM. The alcohol should be incorporated into the membrane to obtained altered sonosensitivity properties; in particular, the alkyl group or hydrocarbon moiety of the alcohol should be embedded in the lipophilic part of the membrane. Thus, membranes coated with an alcohol, like polyvinyl alcohol, are not an essential part of the invention neither is emulgating or solubilising alcohols like e.g. lanolin alcohol and octadecanol.

The particulate material of the invention may further comprise a lipid. Preferably, the lipid is an amphiphilic lipid such as a sphingolipid and/or a glycerol based lipid like e.g. phospholipids. In a preferred embodiment, the amphiphilic lipid is a phospholipid of any type or source. The phospholipid may be saturated or unsaturated, or a combination thereof, although saturated phospholipids are preferred. Typically, the selected phospholipid will have an acyl chain length longer than 12 carbon atoms, more often longer than 14 carbon atoms, and even more often longer than 16 carbon atoms. Preferably, the acyl chain length is within the range 12 to 22 carbon atoms, more preferably within 14 to 20 carbon atoms, and even more preferably 16 to 18 carbon atoms. Acyl chain of different lengths may be mixed in the particulate material of the invention or all acyl chains may have similar or identical length. In one embodiment of the current invention the acyl chain length of the main phospholipid is 18 carbon atoms.

Furthermore, the polar head of the phospholipid may be of any type, e.g. to phosphatidylethanolamine (PE), phosphatidylcholine (PC), phosphatidic acid PA, phosphatidyl serine (PS), or phosphatidylglycerol (PG). In addition, the material of the invention may comprise mixtures of phospholipids with different polar heads. Neutral phospholipid components of the lipid bilayer are preferably a phosphatidylcholine, most preferably chosen from diarachidoylphosphatidylcholine (DAPC), hydrogenated egg phosphatidylcholine (HEPC), hydrogenated soya phosphatidylcholine (HSPC), distearoylphosphatidylcholine (DSPC), dipalmitoylphosphatidylcholine (DPPC) and dimyristoylphosphatidylcholine (DMPC). Negatively charged phospholipid components of the lipid bilayer may be a phosphatidylglycerol, phosphatidylserine, phosphatidylinositol, phosphatidic acid or phosphatidylethanolamine compound, preferably a phosphatidylglycerol like DPPG or a phosphatidylethanolamine like DSPE. In one embodiment of the current invention the main phospholipid is PC and/or PE, more particularly DSPC and/or DSPE.

Components for improving blood circulation time and/or further modulate sonosensitivity may be included in the material, like e.g. polyvinyl alcohols, polyethylene glycols (PEG), dextrans, or polymers. PEG or a derivate thereof, at any suitable concentration, is preferred. However, PEG concentrations are preferably up to 15 mol %, more preferably in the range 3 to 10 mol %, even more preferably in the range 3 to 8 mol %, and most preferably within the range 5.5 to 8 mol %. In preferred embodiments of the current invention the PEG concentration is 3, 8, or 10 mol %. The PEG moiety may be of any molecular weight or type, however, it is preferred that the molecular weight is within the range 350 to 5000 Da, more preferably within 1000-3000 Da. In a preferred embodiment, the molecular weight is 2000 Da. The PEG moiety may be associated with any molecule allowing it to form part of the particulate material. Preferably, the PEG moiety is conjugated to a sphingolipid (e.g. ceramide), a glycerol based lipid (e.g. phospholipid), or a sterol (e.g. cholesterol), more preferably to a ceramide and/or PE, and even more preferably to PE, like DMPE, DPPE, or DSPE. The acyl chain length should be the same as that of the main phospholipid of the membrane. The lipid-grafted PEG is preferably DPPE-PEG 2000 and/or DPPE-PEG 5000. In a particularly preferred embodiment, lipid-grafted PEG is DSPE-PEG 2000.

The material of the invention may be of any size. However, sizes facilitating the so-called enhanced permeability and retention effect (EPRE) are preferred (Maeda H, Matsumura Y, Crit Rev Ther Drug Carrier Syst, 6: 193-210, 1989). Hence, the size of the particulate material used in the invention should be less than 1000 nm, preferably less than 500 nm, more preferably less than 200 nm, more preferably 150 nm or less. In a preferred embodiment, the size falls within the range 50 to 150 nm, more preferably 50 to 95 nm, even more preferably 80 to 90 nm. In a most preferred embodiment, the size is about 85 nm. All size measurements are conducted as is described in the Examples section.

The particulate material may also comprise a sterol, wherein the sterol may be cholesterol, a secosterol, or a combination thereof. The secosterol is preferably vitamin D or a derivate thereof, more particularly calcidiol or a calcidiol derivate. The particulate material may comprises any suitable sterol concentration, preferably cholesterol, depending on the specific particle properties. In general, 50 mol % sterol is considered the upper concentration limit in liposome membranes. Preferably, the cholesterol concentration is within the range 20 to 40 mol %. In embodiments of the current invention, the particulate material comprises 20 or 40 mol % cholesterol.

Furthermore, the particulate material of the invention typically comprises a drug. The drug may be any drug suitable for the purpose. However, anti-bacterial drugs, anti-inflammatory drugs, anti cancer drugs, or any combination thereof is preferred. As the current technology is particularly adapted for treating cancer, anti cancer drugs are preferred. Anti cancer drugs includes any chemotherapeutic, cytostatic or radiotherapeutic drug. It may be of special interest to load the current particulate material with deoxyribonucleic acid (DNA) or ribonucleic acid (RNA), in particular small interfering RNA (siRNA).

The general groups of cytostatics are alkylating agents (L01A), anti-metabolites (L01B), plant alkaloids and terpenoids (L01C), vinca alkaloids (L01CA), podophyllotoxin (L01CB), taxanes (L01CD), topoisomerase inhibitors (L01CB and L01XX), antitumour antibiotics (L01D), hormonal therapy. Examples of cytostatics are daunorubicin, cisplatin, docetaxel, 5-fluorouracil, vincristine, methotrexate, cyclophosphamide and doxorubicin.

Accordingly, the drug may include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, and angiogenesis inhibitors.

The drug may also be alpha emitters like e.g. radium-223 (223Ra) and/or thorium-227 (227Th) or beta emitters. Other alpha emitting isotopes currently used in preclinical and clinical research include astatine-211 (211At), bismuth-213 (213Bi) and actinium-225 (225Ac).

Moreover, the drug may further comprise anti-cancer peptides, like telomerase or fragments of telomerase, like hTERT; or proteins, like monoclonal or polyclonal antibodies, scFv, tetrabodies, Vaccibodies, Troybodies, etc.

More specifically, therapeutic agents that may be included in the particulate material include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, zoledronate, and ELACYT™.

The drug is preferably cyclophosphamide, methotrexate, fluorouracil (5-FU); anthracyclines, like e.g. doxorubicin, epirubicin, or mitoxantrone; cisplatin, etoposide, vinblastine, mitomycin, vindesine, gemcitabine, paclitaxel, docetaxel, carboplatin, ifosfamide, estramustine, or any combination thereof; even more preferably doxorubicin, methotrexate, 5-FU, cisplatin, or any combination thereof. In a preferred embodiment of the current invention the drug is a water soluble drug.

The membrane of the current particulate material may also comprise ligands or antibodies to render active targeting possible. Furthermore, said material may comprise agents, e.g. MRI, X-ray, or optical imaging contrast agents, to make tracking or monitoring possible (See e.g. WO2008033031 or WO2008035985, both incorporated herein by reference).

The particulate material as described anywhere supra may comprise air bubbles of perfluorobutane or perfluoropropane gas, or any non-dissolved gasses, although said material typically will comprise no air bubbles to obtain a small particle size, e.g. lower than 200 nm, more preferably about 100 nm or below. Small size is essential to achieve the so-called EPR effect and consequently passive accumulation in tumour tissue. Preparation of liposomes is well known within the art and a number of methods may be used to prepare the current particles.

Another aspect of the current invention is a method for delivering a drug to a predefined tissue volume comprising administering the particulate material of the invention, as described supra, to a patient in need thereof.

Yet another aspect is a method for treating a disease or condition comprising administering the particulate material of the invention, as described supra, to a patient in need thereof.

The disease to be treated is typically of localised nature, although disseminated disease may also be treated. The disease may be neoplastic disease, cancer, inflammatory conditions, immune disorders, and/or infections, preferably localised variants. The methods described are particularly well suited to treat cancers, in particular solid tumours. Cancers readily available for ultrasound energy are preferred like e.g. cancers of head and neck, breast, cervix, kidney, liver, ovaries, prostate, skin, pancreas, as well as sarcomas. The current sonosensitive particles are well suited to treat all above conditions as they naturally accumulate in such disease volumes.

The drug payload of the sonosensitive material is efficiently released by means of ultrasound. In this way the patient is protected against potential toxic effects of the drug en route to the target tissue and high local concentrations of the drug are obtainable in short time. Hence, the methods supra further comprise the step of exposing the patient to acoustic energy or ultrasound. Preferably, only the diseased volume is exposed to ultrasound, but whole body exposures are also possible. The acoustic energy or ultrasound should preferably have a frequency below 3 MHz, more preferably below 1.5 MHz, more preferably below 1 MHz, more preferably below 0.5 MHz, more preferably below 0.25 MHz, and even more preferably below 0.1 MHz. In preferred embodiments of the current invention, the frequency is 1.17 MHz, 20 kHz or 40 kHz. It should, however, be noted that focused ultrasound transducers may be driven at significantly higher frequencies than non-focused transducers and still induce the current sonosensitive material to release its payload efficiently. Without being limited to established scientific theories, the current inventors believe that ultrasound induced cavitation in the target tissue is the primary physical factor inducing drug release in the present case. A person skilled in the art of acoustics would know that ultrasound at any frequency may induce so-called transient or inertial cavitation.

Furthermore, the current invention comprises the material as described above for medical use.

The current invention also relates to the use of the particulate material as described above for manufacturing a medicament for treating a disease or condition in a patient in need thereof. The use is particularly efficient in treating localised disease, more particularly cancer, immune disorders, inflammatory disease, and/or infective disease. Localised cancers are preferred, as described supra. A particular feature of the current use is the mode of drug delivery: the drug is preferably released from the material of the invention by means of ultrasound. Hence, the drug is administered or activated by means of ultrasound. As noted above, the ultrasound may be of different frequencies, produced by a focused or nonfocused transducer. Notably, the drug may be released by low-frequency nonfocused ultrasound transducers.

The current invention further comprises a composition comprising the above sonosensitive particulate material, as well as a pharmaceutical composition comprising the above sonosensitive particulate material.

Also, the current invention comprises a kit comprising the material of the invention, as well as a process or method of preparing the material of the invention.

Furthermore, the invention comprises a process or method of producing the particulate material of the invention. Said method or process comprising the steps of producing a thin film of the constituents, except membrane embedded alcohol, of the membrane as described above, and then hydrating the film with a suitable hydration liquid containing the alcohol, e.g. hexanol. The method or process may further comprise a freeze-thaw cycle followed by an extrusion process. The drug may be included in the hydration liquid or actively loaded at the end of the process or method. Embodiments of method or process are described in detail in the Examples section.

The current invention also comprises a product produced by the process or method described supra.

### DESCRIPTION OF THE DRAWINGS

FIG. 1. Percent calcein release from liposomes (90 mol % DSPC, 10 mol % DSPE-PEG 2000) with and without hexanol during exposure to 20 kHz ultrasound up to 4 minutes. Closed circles with hexanol, open squares without hexanol.

FIG. 2. Percent calcein release from liposomes (50 mol % DSPC, 10 mol % DSPE-PEG 2000, 40 mol % cholesterol) with and without hexanol during exposure to 20 kHz ultrasound up to 4 minutes. Closed circles with hexanol, open squares without hexanol.

FIG. 3. Percent calcein release from liposomes (62 mol % DSPE, 10% DSPC, 8 mol % DSPE-PEG 2000, 20 mol % cholesterol) with and without hexanol during exposure to 40 kHz ultrasound up to 6 minutes.

FIG. 4. Percent calcein release from liposomes (77 mol % DSPE, 3 mol % DSPE-PEG 2000, 20 mol % cholesterol) with three different nominal concentrations (25, 50, and 75 mM) of hexanol during exposure to 40 kHz ultrasound up to 6 minutes.

### EXAMPLES

The following examples are meant to illustrate how to make and use the invention. They are not intended to limit the scope of the invention in any manner or to any degree.

### Example 1

### Preparation of Liposomes

DSPC and DSPE-PEG 2000 were purchased from Genzyme Pharmaceuticals (Liestal, Switzerland). Cholesterol, calcein, HEPES, sodium azide and sucrose were obtained from Sigma Aldrich. Hexanol was supplied by BDH Chemicals Ltd. (Poole, England).

Calcein carrying liposomes (liposomal calcein) of different membrane composition were prepared using the thin film hydration method (Lasic 1993). The nominal lipid concentration was 16 mg/ml. Liposomes were loaded with calcein via passive loading, the method being well known within the art. The hydration liquid consisted of 10 mM HEPES (pH 7.4) and 50 mM calcein. In liposomes containing hexanol, the hydration liquid was supplemented with a given amount of hexanol 2 days prior to usage in the lipid film hydration step.

The size of the liposomes were made 80-90 nm by extrusion (Lipex, Biomembrane Inc. Canada) at 65° C. (PC liposomes) through polycarbonate (Nuclepore) filters of consecutive smaller size.

Extraliposomal calcein was removed by extensive dialysis. The dialysis was performed by placing disposable dialysers (MW cut off 100 000 D) containing the liposome dispersion, in a large volume of an isosmotic sucrose solution containing 10 mM HEPES and 0.02% (w/v) sodium azide solution. The setup was protected from light and the dialysis ended until the trace of calcein in the dialysis minimum was negligible. The liposome dispersion was then, until further use, stored in the fridge protected from light.

### Example 2

### Characterisation of Liposomes

Liposomes were characterised with respect to key physicochemical properties like particle size, pH and osmolality by use of well-established methodology.

The average particle size (intensity weighted) and size distribution were determined by photon correlation spectroscopy (PCS) at a scattering angle of 173° and 25 deg C. (Nanosizer, Malvern Instruments, Malvern, UK). The width of the size distribution is defined by the polydispersity index. Prior to sample measurements the instruments was tested by running a latex standard (60 nm). For the PCS measurements, 10 μL of liposome dispersion was diluted with 2 mL sterile filtered isosmotic sucrose solution containing 10 mM HEPES (pH 7.4) and 0.02% (w/v) sodium azide. Duplicates were analysed.

Osmolality was determined on non-diluted liposome dispersions by freezing point depression analysis (Fiske 210 Osmometer, Advanced Instruments, MA, US). Prior to sample measurements, a reference sample with an osmolality of 290 mosmol/kg was measured; if not within specifications, a three step calibration was performed.

Duplicates of liposome samples were analysed.

### Example 3

### US Mediated Release Methodology

Liposome samples were exposed to 20 kHz ultrasound up to 4 min in a custom built sample chamber as disclosed in Huang and MacDonald (Huang and Macdonald 2004). The US power supply and converter system was a ‘Vibra-Cell’ ultrasonic processor, VC 750, 20 kHz unit with a 6.35 cm diameter transducer, purchased from Sonics and Materials, Inc. (USA). Pressure measurements were conducted with a Bruel and Kjaer hydrophone type 8103.

The system was run at the lowest possible amplitude at 20% of maximum amplitude. This translates to a transducer input power of 0.9-1.2 W/cm² and a peak-to-peak transducer pressure of about 460 kPa.

For the US measurements, liposome dispersions were diluted in a 1:500 volume ratio, with isosmotic sucrose solution containing 10 mM HEPES (pH 7.4) and 0.02% (w/v) sodium azide. Duplicates were analysed.

The release assessment of calcein is based on the following well-established methodology: Intact liposomes containing calcein will display low fluorescence intensity due to self-quenching caused by the high intraliposomal concentration of calcein (here 50 mM). Ultrasound mediated release of calcein into the extraliposomal phase can be detected by an increase in fluorescence intensity due to a reduced overall quenching effect. The following equation is used for release quantification:

$$\% release = \frac{( {F}_{u} - {F}_{b} )}{( {F}_{T} - {F}_{b} )} \times 100$$

Where F_{b} and Fᵤ are, respectively, the fluorescence intensities of the liposomal calcein sample before and after ultrasound application. F_{T} is the fluorescence intensity of the liposomal calcein sample after solubilisation with surfactant (to mimic 100% release). Studies have shown that the solubilisation step must be performed at high temperature, above the phase transition temperature of the phospholipid mixture. Fluorescence measurements were carried out with a Luminescence spectrometer model LS50B (Perkin Elmer, Norwalk, Conn.) equipped with a photomultiplier tube R3896 (Hamamatsu, Japan). Fluorescence measurements are well known to a person skilled in the art.

### Example 4

### Alcohol Improves the Sonosensitivity of Liposomes

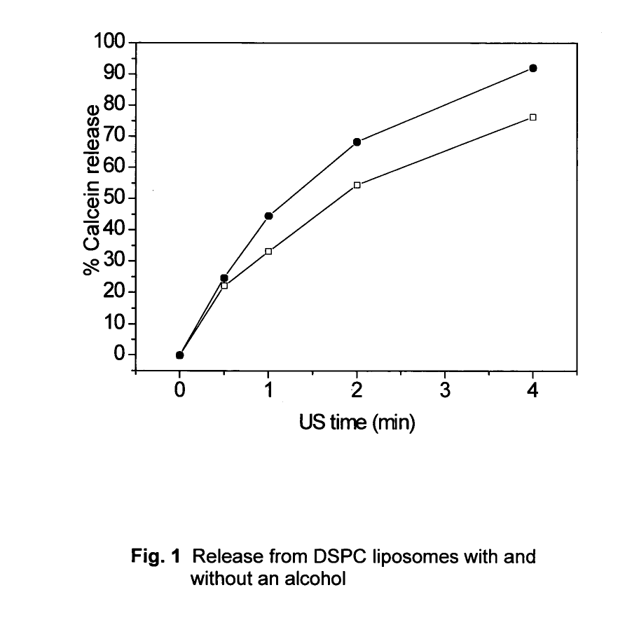

Two liposome formulations, composed of 90 mol % DSPC and 10 mol % DSPE-PEG2000, and containing either hexanol or not were prepared, according to Example 1. For the liposomes containing hexanol, the calcein solution (hydration liquid) was doped with hexanol at 60 mM concentration. The size of the hexanol containing liposomes was measured to 82 nm, while non-hexanol containing liposomes measured 95 nm (see Example 2 for size measurement methodology). The liposomes (diluted 1:500 v/v) were exposed to 20 kHz in the US chamber and the percentage of calcein release was estimated by fluorescence measurements after 0.5, 1, 2 and 4 minutes of ultrasound treatment. FIG. 1 shows that for the formulation containing hexanol (full dots), the sonosensitivity improved yielding an increase in calcein release of 20% (in absolute value) compared to the liposome formulation containing no hexanol (open squares), both after 4 minutes of ultrasound treatment.

### Example 5

### Alcohol Improves the Sonosensitivity of Cholesterol Containing Liposomes

The development of a stable liposome formulation often requires the inclusion of a sterol in the membrane. Also, liposome size is known to affect ultrasound sensitivity. Therefore, the effect of incorporating hexanol on the sonosensitivity was evaluated for similar sized liposomes consisting of 50 mol % DSPC, 10 mol % DSPE-PEG2000 and 40 mol % cholesterol. The liposomes were loaded with calcein as previously described and the size of hexanol and non-hexanol containing liposomes was measured to 88 nm and 89 nm, respectively. For the liposomes containing hexanol, the calcein solution (hydration liquid) was doped with hexanol at 60 mM concentration.

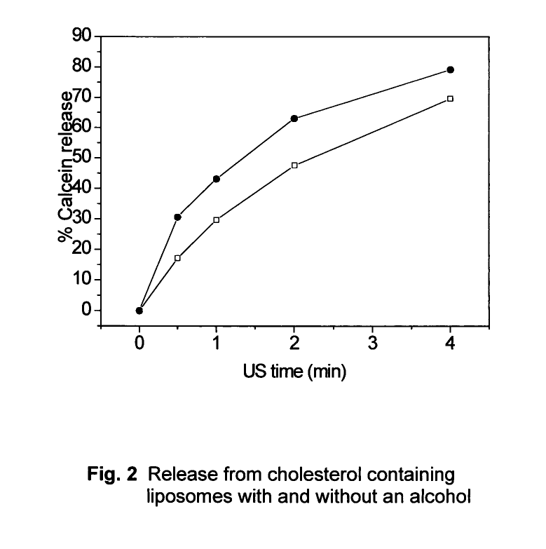

The ultrasound experiment was executed as described above at 20 kHz. Results are shown in FIG. 2. An increase in calcein release of at least 15% (in absolute value) was observed for hexanol containing liposomes compared to liposomes devoid of hexanol (open squares). The beneficial effect of hexanol was seen already at 0.5 min US.

We conclude that that the inclusion of hexanol in the liposome dispersion increases the sonosensitivity of the liposomes.

### Example 6

### Alcohol Improves the Drug Release Properties of PE Liposomes

To evaluate the effect of alcohol on PE liposomal formulations, liposomes composed of 62 mol % DSPE with and without hexanol were investigated. Both formulations further consisted of 8 mol % DSPE-PEG2000, 20 mol % cholesterol, and 10 mol % DSPC. The calcein solution (hydration liquid) contained 50 mM hexanol. Liposomes were prepared and analysed as described supra. The size of liposomes with and without hexanol was 83 nm and 84 nm, respectively. The ultrasound experiments were performed at 40 kHz and the percentage of calcein release was estimated by fluorescence measurements after 0.5, 1, 1.5, 2 and 6 minutes of ultrasound exposure.

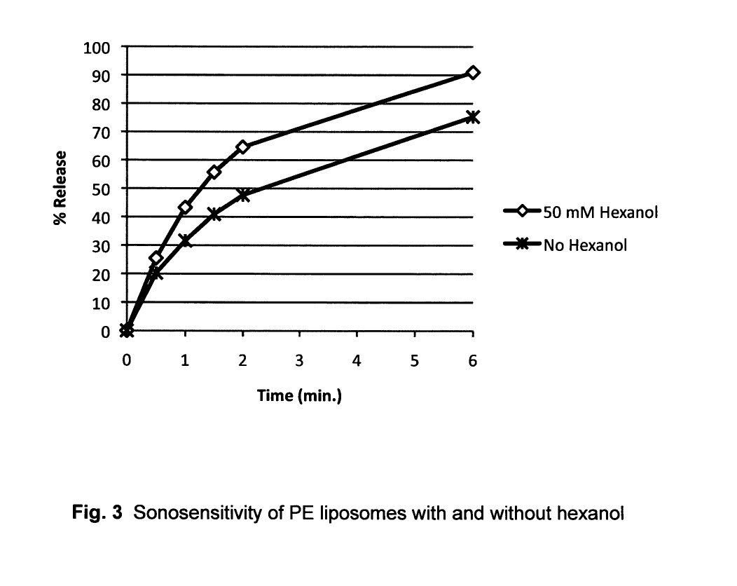

FIG. 3 shows that for the DSPE-based liposomes with hexanol (open diamonds), the sonosensitivity was increased compared to DSPE-based liposomes without hexanol (stars).

We conclude that inclusion of hexanol increases the sonosensitivity and drug release properties of PE-based liposomes.

### Example 7

### Drug Release Properties are Dependent on Alcohol Concentration

To evaluate the effect of different concentrations of alcohol on liposomal formulations, liposomes composed of 77 mol % DSPE with three different nominal concentrations of hexanol were investigated. The tested nominal concentrations were 25, 50 and 75 mM hexanol. All formulations further consisted of 3 mol % DSPE-PEG 2000, and 20 mol % cholesterol. Liposomes were prepared and analysed as described supra. The size of the liposomes prepared initially with 25, 50, and 75 mM hexanol were 84, 88, and 86 nm, respectively. The ultrasound experiments were performed at 40 kHz and the percentage of calcein release was estimated by fluorescence measurements after 0.5, 1, 1.5, 2 and 6 minutes of ultrasound exposure.

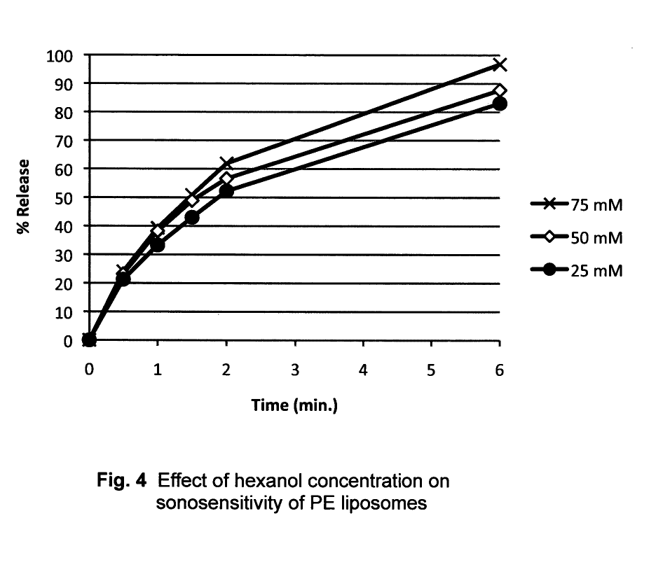

FIG. 4 shows that increasing hexanol concentrations result in liposomes with improved sonosensitivity and drug release properties. We conclude that the sonosensitivity, and consequently the drug release properties, of liposomes vary with membrane-associated alcohol concentration.

### REFERENCES

Aagaard, T., M. kristensen, et al. (2006). “Packing properties of 1-alkanols and alkanes in a phospholipid membrane.” Biophys. Chem. 119: 61-68.
Andresen, T. L., S. S. Jensen, et al. (2005). “Advanced Startegies in Liposomal Cancer Therapy: problems and prospects of active and tumor specific drug release.” Prog. Lipid Res. 44(1): 68-97.
Barry, B. W. (2001). “Novel mechanisms and devices to enable successful transdermal drug delivery.” European Journal of Pharmaceutical Sciences 14.
Drummond, D. C., O. Meyer, et al. (1999). “Optimizing Liposomes for Delivery of Chemotheraoeutic Agents to Solid Tumors.” Pharmacol. Rev. 51(4): 691-743.
Holland, J. W., P. R. Cullis, et al. (1996). “Poly(ethylene glycol)-Lipid Conjugates Promote Bilayer Formation in Mixtures of Non-Bilayer-Forming Lipids.” Biochemistry 35: 2610-2617.
Huang, S, and R. C. Macdonald (2004). “Acoustically active liposomes for drug encapsulation and ultrasound-triggered release.” Biochim. Biophys. Acta 1665: 134-141.
Larina, I. V., B. M. Evers, et al. (2005). “Enhancement of drug delivery in tumors by using interaction of nanoparticles with ultrasound radiation.” Technol Cancer Res Treat 4(2): 217-226.
Larina, I. V., B. M. Evers, et al. (2005). “Optimal drug and gene delivery in cancer cells by ultrasound-induced cavitation.” Anticancer Res 25(1A): 149-156.
Lasic, D. D. (1993). Liposomes from Physics to Applications. Amsterdam, Amsterdam Elsevier Science Publishers BV.
Lee, A. G. (1976). “Interactions between anesthetics and lipid mixtures. Normal alcohols.” Biochemistry 15: 2448-2454.
Lin, H. Y. and J. L. Thomas (2003). “PEG-Lipids and Oligo(ethylene glycol) Surfactants Enhance the Ultrasonic Permeabilizability of Liposomes.” Langmuir 19(4): 1098-1105.
Maeda H, Matsumura Y. Tumoritropic and lymphotropic principles of macromolecular drugs. Crit Rev Ther Drug Carrier Syst, 1989; 6: 193-210.
Myhr, G. and J. Moan (2006). “Synergistic and tumor selective effects of chemotherapy and ultrasound treatment.” Cancer Lett. 232: 206-213.
Pitt, W. G., G. A. Husseini, et al. (2004). “Ultrasonic drug delivery—a general review.” Expert Opin Drug Deliv 1(1): 37-56.
Rowe, E. S. and J. M. Campion (1994). “Alcohol Induction of Interdigitation in Distearoylphosphatidylcholine: Fluorescence Studies of Alcohol Chain Length Requirements.” Biophys. J. 67: 1888-1895.
Thewalt, J. L. and R. J. Cushley (1987). “Phospholipid/cholesterol membranes containing n-alkanols: a 2H-NMR study.” Biochim. Biophys. Acta 905: 329-338.
Tierney, K. J., D. E. Block, et al. (2005). “Elasticity and Phase Behavior of DPPC Membrane Modulated by Cholesterol, Ergosterol, and Ethanol.” Biophys. J. 89: 2481-2493.
WO 94/28873, Unger et al.
WO A1 9428874, Unger et al.
U.S. Pat. No. 5,770,222, Unger et al.

1. A method for treating a disease or condition, comprising:
administering a particulate material comprising an alcohol and a medicament to a patient in need thereof, wherein the alcohol is propanol, butanol, hexanol, heptanol, or octanol, or any combination thereof, and wherein the medicament is administered by means of ultrasound.

2. The method according to claim 1, wherein the alcohol is hexanol, heptanol, or octanol, or any combination thereof.

3. The method according to claim 2, wherein the alcohol is hexanol.

4. The method according to claim 1, wherein said particulate material further comprises a phospholipid.

5. The method according to claim 4, wherein the phospholipid is phosphatidylcholine (PC) and/or phosphatidylethanolamine (PE).

6. The method according to claim 1, said particulate material further comprising a polyethyleneglycol or a derivative thereof.

7. The method according to claim 1, wherein the particulate material has a size that is below 200 nm.

8. The method according to claim 1, wherein the particulate material is an emulsion, microemulsion, liquid crystalline particles or liposome.

9. The method according to claim 8, wherein the particulate material is a liposome.

10. The method according to claim 1, wherein the particulate material does not comprise any non-dissolved gasses.

11. The method according to claim 1, wherein the disease or condition is cancer, inflammation or infection.

12. An ultrasound sensitive particulate material comprising an alcohol, wherein said alcohol is propanol, butanol, hexanol, heptanol, or octanol, or any combination thereof.

13. The material of claim 12, wherein the alcohol is hexanol, heptanol, or octanol, or any combination thereof.

14. The material of claim 13, wherein the alcohol is hexanol.

15. The material of claim 12, further comprising a phospholipid.

16. The material of claim 12, further comprising phosphatidylcholine (PC) and/or phosphatidylethanolamine (PE).

17. The material of claim 12, further comprising a polyethyleneglycol or a derivative thereof.

18. The material of claim 12, wherein the particulate material is a liposome.

19. The material of claim 12, wherein the particulate material has a size that is less than 200 nm.

20. The material of claim 19, wherein the size is less than 150 nm.

21. The material of claim 12, not comprising any non-dissolved gasses.

22. The material of claim 12, wherein said material is suitable for medical use.
